(19) United States
(12) Patent Application Publication
(10) Pub. No.: US 2007/0166313 A1
(43) Pub. Date: Jul. 19, 2007

(54) Administration of gamma globulins to treat cancer

(76) Inventors: Yehuda Shoenfeld, Ramat-Gan (IL); Miri Blank, Tel-Aviv (IL)

Correspondence Address: PEARL COHEN ZEDEK LATZER, LLP, 1500 BROADWAY 12TH FLOOR, NEW YORK, NY, 10036, US

(21) Appl. No.: 11/606,364

(22) Filed: Nov. 30, 2006

Related U.S. Application Data
(63) Continuation-in-part of application No. 09/405,050, filed on Sep. 27, 1999, now abandoned.
Application No. 09/405,050 is a continuation-in-part of application No. 08/487,803, filed on Jun. 7, 1995, now Pat. No. 5,965,130.
Application No. 08/487,803 is a continuation of application No. 08/340,094, filed on Nov. 15, 1994, now Pat. No. 5,562,902.
Application No. 08/340,094 is a continuation-in-part of application No. 08/212,361, filed on Mar. 14, 1994, now abandoned.

(51) Int. Cl. A61K 39/395

(52) U.S. Cl. 424/155.1

(57) ABSTRACT

This invention relates to cancer therapy and in particular to the administration of gamma globulins to inhibit both primary tumor and metastasis and augment treatment of primary cancerous tumors. In accordance with this invention, the treatment of various cancerous diseases is accomplished by administering a preparation containing intact gamma globulins or fragments thereof.

### CROSS REFERENCE TO RELATED APPLICATIONS

This is a continuation-in-part of U.S. patent application Ser. No. 09/405,050, filed Sep. 27, 1999, which is a continuation-in-part of U.S. patent application Ser. No. 08/487,803, filed Jun. 7, 1995 now U.S. Pat. No. 5,965,130, which is a continuation of U.S. patent application Ser. No. 08/340,094, filed Nov. 15, 1994 now U.S. Pat. No. 5,562,902, which is a continuation-in-part of U.S. patent application Ser. No. 08/212,361, filed Mar. 14, 1994 now abandoned, all which are incorporated herein by reference in their entirety.

### FIELD OF THE INVENTION

The present invention relates to cancer therapy and in particular to the administration of gamma globulins (IVIG) to inhibit both primary tumors and metastases and augment treatment of primary cancerous tumors. In accordance with this invention, the treatment of various cancerous diseases is accomplished by administering a preparation containing intact gamma globulins or fragments thereof.

### BACKGROUND OF THE INVENTION

The formation of metastases of malignant tumors, initiated from a primary tumor at more or less remote locations of the body, is one of the most serious effects of cancer and one for which a satisfactory treatment protocol is currently unavailable. Cancer tumor metastasis is responsible for most therapeutic failures when the disease is treated, as patients succumb to the multiple tumor growth.

The extent to which metastases occur vary with the individual type of tumor. Melanoma, lymphoma, breast cancer, lung cancer, colon cancer and prostate cancer are among the types of cancers that are particularly prone to metastasize. When metastasis takes place, the secondary tumors can form at a variety of sites in the body, with lungs, liver, brain and bone being the more common sites.

The currently available methods of cancer therapy such as surgical therapy, radiotherapy, chemotherapy and other immunobiological methods have either been unsuccessful in preventing metastasis or these methods give rise to serious and undesirable side effects.

In many clinically diagnosed solid tumors (in which the tumor is a localized growth) surgical removal is considered the prime means of treatment. However, many times after surgery and/or after some delay period, the original tumor is observed to have metastasized so that secondary sites of cancer invasion have spread throughout the body and the patient subsequently dies of the secondary cancer growth. In other embodiments surgical removal of the tumor is not feasible because of the location of the tumor (for example certain areas in the brain) and radiation, chemotherapy or other immunobiological methods are the sole alternatives.

Reports indicate that in individuals with resectable tumors, primary tumor growth or local recurrence is not often the cause of death. Instead, at present, nearly 40% of cancer victims with operable tumors ultimately succumb to metastatic disease following surgery.

Metastasis is a constant occurrence in some tumors. However, many times metastasis is triggered by the surgery itself. During the course of surgery malignant cells may become dislodged from the tumor mass and enter the circulatory system thus increasing the chance of metastasis.

Although chemotherapy is widely used in the treatment of cancer, it is a systemic treatment based usually on the prevention of cell proliferation. Accordingly, chemotherapy is a non-specific treatment modality affecting all proliferating cells, including normal cells, leading to undesirable and often serious side effects such as immunosuppression, pancytopenia (growth inhibition of bone marrow cells with anemia, thrombocytopenia, and leukopenia), diarrhea, nausea or alopecia (hair loss).

Generally, the existing systemic treatments have, quite often, proven to have little effect on micrometastases already residing in remote organs (lung, liver, bone marrow or brain), and they are not very effective in preventing the dissemination of the tumor to other tissues.

Therefore, the need exists for methods for inhibiting tumor metastasis. In particular, methods which inhibit (micro)metastasis without causing serious side effects are much desired.

### SUMMARY OF THE INVENTION

In one embodiment, the invention provides a method for inhibiting colon cancer, or its metastasis in a subject comprising the step of administering to the mammal a preparation of IVIG or fragments thereof, thereby preventing tumor cell proliferation and invasiveness.

In another embodiment, provided herein, is the use of IVIG or fragments thereof in a composition for inhibiting colon cancer, or its metastases in a subject.

### BRIEF DESCRIPTION OF THE DRAWINGS

FIG. 1 shows inhibitory effect of IVIG on CT26 cell proliferation (time and dose response curve). Murine colon carcinoma CT26 cells were cultivated in the 96-well plate with different concentrations of IVIG for 24-, 48- or 72 hours. The rate of cell proliferation was assessed by the XTT proliferation assay kit as described in “Materials and Methods”. Data are expresses as percentage inhibition of cell proliferation (the mean of three separate experiments in duplicate wells). Legend: *, p<0.05; **, p<0.01; ***, p<0.001 versus wells without IVIg; bars represent the standard deviation (SD)

FIG. 2 shows effect of IVIG on CT26 cancer cell invasion in a dose and time dependence study: Murine colon carcinoma CT26 cells (2.5×10⁴ cells/well) were added to the upper wells of a 12-well Transwell chamber and incubated in the presence of two different concentrations of IVIg. Migration across a matrigel-coated membrane was assessed after either 48- or 72 hours by the XTT proliferation assay and measurements of absorbance at 450 nm. Data are expressed as percentage inhibition, the mean of three separate experiments in duplicate wells ±SD. Student's t-test was used to assess quantitative differences between control and IVIG-treated wells.

FIGS. 3 and 4 show the inhibitory effect of IVIG on metastates to the lungs. Lungs were weighed one month following inoculation of mice with murine colon carcinoma CT26 cells. CT26 tumor cells (2×10⁴) were administrated intravenously. At the same time and 14 days after the intrvanous injection of CT26 either maltose or different concentrations of IVIG were administrated via intravenous route. Thirty days after tumor injection, the lungs were harvested and evaluated for lung weight and examined morphologically. Legend: *, p<0.05; **, p<0.01; ***, p<0.001 compared to that of untreated control mice.

FIG. 4 shows upression of lung metsastases by IVIG: Lungs were examined one month following inoculation of mice with murine colon carcinoma CT26 cells. CT26 tumor cells (2×10⁴) were administrated intravenously. At the same time and 14 days after the intrvanous injection of CT26 either maltose or different concentrations of IVIG were administrated via intravenous route. Thirty days after tumor injection, the lungs were harvested and examined morphologically after staining with India Ink or H&E. Lungs were inspected for the presence, number and size of metastatic foci using ex vivo staining with India ink and H & E staining of histological slides. Photographs of the lung of each group are shown. The experiment was repeated three times with similar results.

### DETAILED DESCRIPTION OF THE INVENTION

Definitions

As used herein, “gamma globulin” is the serum globulin fraction that is mainly composed of IgG antibody molecules.

As used herein, “IVIG” or “intravenous immunoglobulins” refers to gamma globulin preparations suitable for intravenous use, such as those IVIG preparations commercially available from several sources.

As used herein, “fragments” of IVIG or gamma globulin are portions of intact immunoglobulins such as Fc, Fab, Fab′, F(ab′)₂ and single chain immunoglobulins.

“Metastasis”, as used herein, is defined as the migraton or transfer of malignant tumor cells, or neoplasms, via the circulatory or lymphatic systems or via natural body cavities, usually from the primary focus of tumor, cancer or a neoplasia to a distant site in the body, and the subsequent development of one or a plurality of secondary tumors or colonies thereof in the one new or the plurality of new locations. In another embodiment, “metastases” means the secondary tumors or colonies formed as a result of metastasis and encompasses micro-metastases.

As used herein, “inhibition of metastasis” is defined as preventing or reducing the development of metastases.

“Intracavitary administration”, as used herein, refers in one embodiment to administering a substance directly into a body cavity of a subject. Such body cavities include the peritoneal cavity, the pleural cavity and cavities within the central nervous system (intrathecal).

“Intravascular adminsitration” as used herein, refers in one embodiment to administering a substance directly into circulatory system of a subject. The circulatory system is comprised of the venous (veins) and arterial (arteries) systems.

The term “about” as used herein refers to quantitative terms plus or minus 5%, or in another an embodiment plus or minus 10%, or in yet another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

Gammaglobulins suitable for intravenous administration are commonly referred to as Intravenous Immunoglobulins (IVIG) and are commercially available from several sources. The commercially available IVIG preparations contain mainly IgG molecules. WIG has been used in replacement therapy in primary immunodeficiency syndromes and in secondary immunodeficiencies as well as for the prevention and treatment of infectious diseases. Furthermore, IVIG has also been used for immune modulation of patients with autoimmune and immune-complex diseases (See, Martha M. Eibl, “Intravenous Immunoglobulin: A Review”, Immunodeficiency Reviews, 1Suppl., pp. 1-42 (1989)).

According to a National Institutes of Health (NIH) Consensus Conference report, the incidence of adverse side effects associated with IVIG use in humans, used at dosage regimens comparable to the ones contemplated by the present invention, is usually less than 5% with most of those reactions being “mild and self-limited.” The report adds that “severe reactions occur very infrequently and usually do not contraindicate further IVIG therapy.” The NIH report also notes that “neither HIV nor hepatitis B infection has been transmitted to recipients of products currently licensed in the United States.” NIH Consensus Conference, “Intravenous Immunoglobulin: Prevention and Treatment of Disease”, JAMA, 264, pp. 3189-3193 (1990).

The present invention stems from our discovery that IVIG as a whole molecule or the F(ab′)₂ fragment by itself, is extremely useful for the treatment of cancerous diseases in murine and rat models.

The gamma globulin preparations that may be used according to the present invention include commercially available preparations of intact IVIG and preparations of the Fc, F(ab′)₂ fragments of IVIG or their combination. Recombinantly produced gamma globulins and their fragments may also be used according to this invention. The use of recombinant single chain antibodies is also envisioned.

The dosage of IVIG and the method of administration will vary with the severity and nature of the particular condition being treated, the duration of treatment, the adjunct therapy used, the age and physical condition of the subject of treatment and like factors within the specific knowledge and expertise of the treating physician. However, single dosages for intravenous and intracavitary administration can typically range from 400 mg to 2 g per kilogram body weight, preferably 2 g/kg (unless otherwise indicated, the unit designated “mg/kg” or “g/kg”, as used herein, refers to milligrams or grams per kilogram of body weight). The preferred dosage regimen is 400 mg/kg/day for 5 consecutive days per month or 2 g/kg/day once a month. The IVIG, according to the present invention, was found to be effective in inhibiting metastasis in animal models when administered by intravenous or intraperitoneal injection and in the dose range of 500-1000 mg/kg/week.

In another embodiment of this invention, the IVIG preparation is administered via the subcutaneous route. The typical dosage for subcutaneous administration can range from 4 mg to 20 mg/kg body weight. The IVIG according to the present invention was found to be effective in inhibiting metastasis in mice when administered subcutaneously in the dose 200 g/mouse. According to the present invention IVIG may be administered as a pharmaceutical composition containing a pharmaceutically acceptable carrier. The carrier must be physiologically tolerable and must be compatible with the active ingredient. Suitable carriers include, sterile water, saline, dextrose, glycerol and the like. In addition, the compositions may contain minor amounts of stabilizing or pH buffering agents and the like. The compositions are conventionally administered through parenteral routes, with intravenous, intracavitary or subcutaneous injection being preferred.

The intravenous immunoglobulins administered according to the present invention act as antimetastatic agents resulting in the reduction of tumor colony number as well as tumor colony size. They can also act prophylactically i.e., to prevent metastasis of tumors. The intravenous immunoglobulins according to this invention may also be used to reduce the size of the primary tumor.

The treatment described in the present invention may also be used either preceding or subsequent to a surgical procedure to remove the primary tumor. Frequently, metastasis of tumor cells will occur as a result of the physical manipulation of the tumor during surgery. However, the use of the treatment described in the present invention in conjunction with (i.e., before, during and/or after) surgery will reduce the risk of metastasis and consequently this combination of methods would be a more attractive treatment option for the complete elimination of cancerous tumors.

Similarly, other treatment modalities such as chemotherapy, radiation therapy and immunotherapy may also be used in conjunction with (i.e., before, after or at the same time as) the methods of the present invention.

Although not wishing to be bound by any particular theory, intravenous immunoglobulins inhibit metastasis according to one or more embodiment of the following mechanisms.

It is known that tumor metastasis occurs following a detachment of single cancerous cells from the tumor, their migration to adjacent or distal tissues, and their seeding and homing in the new organ. The migration process takes place through adhesion molecules which enable the tumor cells to adhere to the blood vessel wall, to penetrate the blood stream and then to emerge and seed in another tissue. In one embodiment, when whole IVIG or the F(ab′)₂ fragments of IVIG are administered, they interfere with the binding of adhesion molecules responsible for the transmission of the tumor cell to and from the blood vessel, and thus prevent the dissemination of the tumor cells to other tissues in the body.

In another embodiment the presence of antibodies or anti-idiotypes in the IVIG mixture bind to the tumor cells and induce their lysis in the presence of complement or enhance entrapment of the tumor cells by Fc receptors on the reticuloendothelial system (RES).

In one embodiment, intravenous immunoglobulins reduce or prevent metastasis by increasing the efficiency of the immune system through inducing the secretion of cytokines such as tumor necrosis factor and γ-interferon.

In the treatment of lymphoma, recent experiments indicate that IVIG acts to induce apoptosis (programmed cell death) in a T-cell lymphoma cell line in vitro.

The effect of IVIG on the dissemination of tumors according to the present invention is demonstrated by the following examples carried out in murine and rat models of melanoma, sarcoma and lymphoma. Additionally, we also present clinical data of a representative human melanoma patient treated with IVIG. These examples are set forth so that this invention may be better understood and are not to be construed as limiting its scope in any manner.

In one embodiment, the compositions and methods described herein are useful in the treatment of colon carcinoma or its metastases. According to this aspect of the invention and in one embodiment, provided herein is a method for inhibiting colon cancer, or its metastases in a subject comprising the step of administering to the mammal a preparation of IVIG or fragments thereof, thereby preventing tumor cell proliferation and invasiveness.

In one embodiment, the term “antibody” includes complete antibodies (e.g., bivalent IgG, pentavalent IgM) or fragments of antibodies which contain an antigen binding site in other embodiments. Such fragments include in one embodiment Fab, F(ab′)₂, Fv and single chain Fv (scFv) fragments. In one embodiment, such fragments may or may not include antibody constant domains. In another embodiment, Fab's lack constant domains which are required for Complement fixation. ScFvs are composed of an antibody variable light chain (V_{L}) linked to a variable heavy chain (V_{H}) by a flexible hinge. ScFvs are able to bind antigen and can be rapidly produced in bacteria. The invention includes antibodies and antibody fragments which are produced in bacteria and in mammalian cell culture. An antibody obtained from a bacteriophage library can be a complete antibody or an antibody fragment. In one embodiment, the domains present in such a library are heavy chain variable domains (V_{H}) and light chain variable domains (V_{L}) which together comprise Fv or scFv, with the addition, in another embodiment, of a heavy chain constant domain (C_{H1}) and a light chain constant domain (C_{L}). The four domains (i.e., V_{H}-C_{H1} and V_{L}-C_{L}) comprise an Fab. Complete antibodies are obtained in one embodiment, from such a library by replacing missing constant domains once a desired V_{H}-V_{L} combination has been identified.

Antibodies of the invention can be monoclonal antibodies (mAb) in one embodiment, or polyclonal antibodies in another embodiment. Antibodies of the invention which are useful for the compositions, methods and kits of the invention can be from any source, and in addition may be chimeric. In one embodiment, sources of antibodies can be from a mouse, or a rat, a plant, or a human in other embodiments. Antibodies of the invention which are useful for the compositions, and methods of the invention have reduced antigenicity in humans (to reduce or eliminate the risk of formation of anti-human antibodies), and in another embodiment, are not antigenic in humans. Chimeric antibodies for use the invention contain in one embodiment, human amino acid sequences and include humanized antibodies which are non-human antibodies substituted with sequences of human origin to reduce or eliminate immunogenicity, but which retain the antigen binding characteristics of the non-human antibody.

In one embodiment, the preparation of IVIG or fragments thereof used in the methods and compositions described herein is administered intravenously, intracavitarily, intratumorally or subcutaneously, at a dosage of between about 0.1 g/kg/month to about 2 g/kg/month.

A person skilled in the art would readily recognize that the mode of administration, and the dosage will depend on many factors, such as inter-alia the stage of cancer of the subject, or the extent of metastases, as well as other factors in certain embodiments.

The active agent is administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual subject, the site of delivery, the method of administration and other factors known to practitioners. Examples of techniques and protocols can be found in Remington's Pharmaceutical Sciences.

Alternatively, targeting therapies may be used in another embodiment, to deliver the active agent more specifically to certain types of cell, by the use of targeting systems such as antibodies or cell specific ligands. Targeting may be desirable in one embodiment, for a variety of reasons, e.g. if the agent is unacceptably toxic, or if it would otherwise require too high a dosage, or if it would not otherwise be able to enter the target cells.

In one embodiment, the colon carcinoma treated by the methods and compositions described herein, is metastasizable to an organ selected from the group of lung, liver, brain or bone.

In one embodiment, metastases are the leading cause of treatment failure and death of patients affected by colon cancer, which makes them a major therapeutic target. The metastatic process comprises in another embodiment, a series of complex interactions between cancerous cells and host cells or tissues. A cell originating from a colon carcinoma site must undergo several modifications to become metastatic. These include loss of adhesion with surrounding cells in one embodiment, or migration towards vessels, destruction of the basement membrane, passage in the blood stream and escape from the immune system, or their combination in other embodiments. The cells must then arrest and extravasate into the target tissue, and growth in this tissue where a neoangiogenesis leads to its blood supply. Accordingly and in another embodiment, the methods and compositions provided herein are effective in the treatment of metastases of colon cancer in a subject.

In another embodiment, the methods and compositions provided herein, are effective in the prevention or inhibition of proliferation, invasion, metastatic capacity or their combination, of colon cancer cells.

The following examples are presented in order to more fully illustrate the preferred embodiments of the invention. They should in no way be construed, however, as limiting the broad scope of the invention.

### EXAMPLES

Materials and Methods:

Tumor Cells

Tumor cell lines from murine origin were used. The cell lines included: MCA-105, a methylcholanthrene-induced sarcoma of C57BL6J origin and B16-F10 melanoma cells (both lines were purchased from American Type Tissue Culture Collection, Rockville, Md.). The cells were routinely maintained in RPMI medium containing 10% fetal calf serum. Twice a week the cells were transferred to a freshly prepared medium

Experimental Animal Models

2-3 months old C57BL/6J mice were used during the study. To examine the efficacy of gamma globulin in vivo, 2 types of solid tumors were induced in C57BU6J mice, e.g. sarcoma (MCA-105 (and melanoma (B16nF10). The tumor cells were induced either by intravenous (IV(injection which led to their seeding and lodging in the lung or by intraperitoneal)IP) induction where the cancerous cells developed local lesions in the peritoneum. Some of the mice were sacrificed following 3-5 weeks and examined for metastatic foci in the lungs or spread of tumors in the peritoneum. In another group of mice, survival time was observed. Mice that were injected with tumor cells by IV mode were treated by IV infusion of gamma globulin, whereas in animals in whom the tumor was induced directly in the peritoneum, the gamma globulin preparation was administered through IP injection.

Gamma Globulin Preparations

Gamma Globulin Preparations: Human gamma globulin suitable for intravenous use (IVIG) was obtained from Miles Inc. (Biological Products Division, West Haven, Conn.). A 5% solution (5 gr in 100 ml diluent; Catalogue No. 0640-12) was used for all experiments.

Unless otherwise indicated, the volume of IVIG inoculated was 500 μl. per animal on each treatment which amounted to 25 mg of gamma globulin per animal. Other preparations used were a whole molecule human gamma globulin or an F(ab′)₂ fragment both purchased from Jackson Immunoresearch Laboratories, Inc., West Grove, Pa., (Code Numbers 009-000-003 and 009-000-006 respectively). These latter preparations are different from the one obtained from Miles Inc. in that they are prepared from a donor pool of 30 whereas the Miles preparation is from a donor pool of 3000 or more individuals.

### Example 1

### Effect of Gamma Globulin on the Development of Metastatic Melanoma in C57BL/6J Mice

An experimental model for metastatic melanoma was established using the B16-F10 mouse melanoma cell line. The induction of the melanoma was carried out by IV injection of the tumor melanoma cells which are subsequently seeded in the mice lung and form black metastatic foci. Approximately 24 days following tumor inoculation, the mice die.

In the present experiment the mice were injected either with 2×105 tumor cells or with 5×1O5 cells and were treated intravenously with IVIG (Miles). The mice were sacrificed on day 18 and the efficacy of the treatment was determined by counting the number of the black metastatic foci in the lungs of the animals.

Inoculation of Mice with 2×105 B16-F10 Melanoma Cells.

20 mice were IV injected with 2×105 melanoma cells and were divided into 4 groups: (a) Control group, mice inoculated with tumor cells only; (b) The mice treated with one IV injection of IVIG on day 0 (the day of tumor administration); (c) The mice treated 2 times, on day 0 and on day 4; and (d) The mice treated 3 times on days 0, 4 and 9.

The mice were sacrificed on day 18 and the number of the metastatic foci in the lungs was evaluated. Table I summarizes the results. One treatment reduced the number of metastatic foci by 80%, while no foci could be detected following two or three treatments.

TABLE I

| GROUP | NO. OF FOCI |
|---|---|
| Control | 20 ± 4 |
| 1 Treatment | 4 ± 2 |
| 2 Treatments | 0 |
| 3 Treatments | 0 |

Black metastatic foci are seen in the control group, less foci in the group that was treated with 1 injection of IVIG and none is seen in the lungs that were derived from mice treated by two or three injections of IVIG.

B. Inoculation of Mice with 5×105 B16-F10 Melanoma Cells

In order to explore whether IVIG is capable of preventing metastasis when a larger mass of melanoma was involved, the following experiment was conducted. Mice were injected with an increased number of tumor cells (5×10⁵) and divided into 2 groups: (a) Control group, inoculated with tumor cells only; and (b) mice treated with 2 IV injections of IVIG on day 0 and on day 8 following tumor inoculations.

On day 18, the mice were sacrificed. Evaluation of the lung metastatic foci revealed a marked decrease in their number in the IVIG treated group. The results are summarized in Table II.

TABLE II
Reduction in number of metastatic foci in the lungs of mice
injected with 5 × 10⁵ B-16 F10 melanoma cells
and then treated with IVIG

| GROUP | NO. OF FOCI |
|---|---|
| Control | 165 ± 13.4 |
| Two Treatments | 16.3 ± 3.9 |

There is about a 90% reduction in the number of foci in the treated mice when compared to the control group. These results show that WIG is capable of inhibiting metastatic spread of melanoma even when a larger tumor mass is involved.

C. Effect of Gamma Globulin on the Survival of Melanoma Bearing Mice

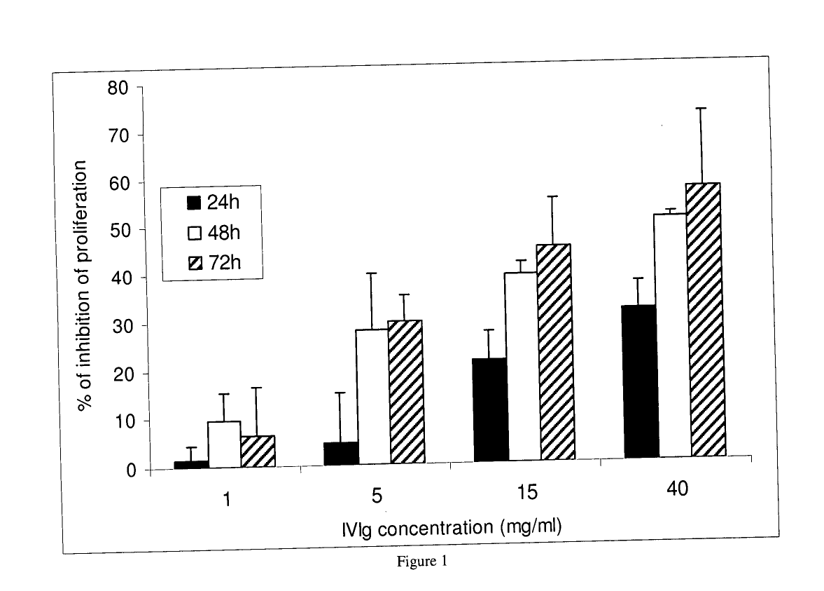

4×10⁵ B16-F10 cells were injected W to C57BU6J mice. The mice were treated from day 0 and every 7^{th} day thereafter with 500 μl Miles IVIG. 24 days following the inoculation of the tumor, the mice from the control group began to die. The results are summarized in FIG. 1. While on day twenty six 100% of the control mice were dead, 100% of the IVIG treated mice were alive. On day 40, of the treated mice were still alive.

### Example 2

### Effect of Gamma Globulin on the Development of MCAn105 Sarcoma in C57BL/6J Mice

MCA-105 cells are derived from mice that developed tumors following methylcholanthrene administration. Two types of experiments were carried out using these cells:

1. Tumor Induction by IV Infusion:

2.5×10⁵ MCA-105 cells were injected IV to the tail vein of C57BL/6J mice. One group of mice was treated by IV infusion of IVIG on days 0, and 14 and the mice belonging to the control group were injected on the same days with phosphate buffered saline (PBS).

40 days later the mice were sacrificed and their lungs were evaluated for tumor lesions. The lungs of the control group of mice were much larger than those of the IVIG treated animals and were covered with metastatic foci which appeared as white “blebs”.

2. Tumor Induction by IP Infusion:

2.5×10⁵ MCA-105 cells were injected IP to C57BU6J mice. The mice were treated with IVIG from day 0 and every 7th day thereafter till they were sacrificed on day 48. Large tumor foci were observed in the peritoneum in the control group of mice whereas in the treated animals only few small foci were seen.

### Example 3

### Comparison of the Effect of Intact Gamma Globulin and F(ab′)₂ Fragment on the Development of B-16 Melanoma in C57BL/6J Mice

Mice were IV inoculated with 2.5×10⁵ B16-F10 melanoma cells. The mice were divided into 3 groups:
a) A control group;
b) mice treated with whole molecule IVIG (Jackson Immunoresearch Laboratories Inc.); 5 mg in a volume of 330 μl was injected IV on days 0, 3, 7 and 12; and
c) a group of mice that was treated with 5 mg of F(ab′)₂ fragment of IVIG (Jackson) in a volume of 500 μl on the same days as in (b).

The mice were sacrificed on day 17 and black metastatic foci were counted in the lung. In the control group 160±18 metastatic foci were counted in comparison to 68±12 in the group treated with the preparation of intact IVIG, and 13+4 foci in the mice treated with the preparation of F(ab′)₂ fragments of IVIG. This result indicates that both intact IVIG and their F(ab′)₂ fragments are effective in inhibiting metastases. The observed difference in the effectiveness between the intact IVIG and the F(ab′)₂ preparation is probably due to the difference in the specific activities of the two solutions used. The difference between the results of Example 1 where the mean number of metastatic foci was 4 when intact IVIG were administered (Table I) and the present example where the mean number of foci was 68 is probably due to the fact that in Example 1,25 mg of whole IVIG was injected on day 0, whereas in this example only 5 mg was injected on day 0.

### Example 4

### Effect of Gamma Globulin in the Inhibition of Metastasis of Melanoma Following Surgical Removal of Primary Tumor

C57BL6J mice were injected in the foot pad with 2.5×10³ melanoma cells. After 21 days the leg in which tumor developed was amputated. On the same day the mice were divided into two groups, one group was treated by intravenous injection of 25 mg IVIG (Sandoz Pharma. Ltd., Basle, Switzerland; Lot-4.372.256.0) and the other group with phosphate buffered saline (PBS). Ten days later, the mice were examined for signs of tumor development. 60% of the control group developed tumor with a mean size of 3±0.8 cm. Those mice died during the first month following amputation.

In the IVIG treated group, only 14% of the mice developed tumor (mean size 2.7±1.2 cm). These mice also died during the first month following amputation. The remaining 86% of the IVIG treated mice did not develop tumor and were still alive 45 days following surgery.

### Example 5

### Effect of Low Dose, Subcutaneous Administration of Gamma Globulin in the Inhibition of Melanoma Metastasis

C57BL6J mice were injected intravenously with 2.5×10⁵ B16 melanoma cells per mouse. Immediately thereafter, the mice were administered IVIG preparations via the subcutaneous route in the chest area. Four groups of mice (20 mice/group) received 200 g/mouse of one of the following commercially available IVIG preparations obtained from Baxter (Gammagard S/D 2.; Lot-93H23AB12C), Isiven (Isiven V. I. 2. lot-IS238C6193V), Miles (Gamimmune N 5%; Lot-640N023) and Sandoz (Lot-4. 372.256.0). A fifth group of mice receiving intravenous PBS administration acted as the control group. The mice were sacrificed 18 days later and their lungs examined for the presence of metastatic foci. The following table depicts the results.

TABLE III

| GROUP | NO. OF FOCI | % INHIBITION |
|---|---|---|
| Control | 50 | — |
| Baxter | 18.6 | 62.8 |
| Miles | 28.5 | 43.0 |
| Isiven | 18.1 | 63.8 |
| Sandoz | 27.9 | 44.2 |

As shown above, low dose, subcutaneous IVIG administration inhibited melanoma metastasis by an average of 53.45% when compared to mice treated with PBS.

### Example 6

### Protocol for Use of Gamma Globulin to Inhibit Metastasis in Human Cancer Patients

IVIG preparations are parenterally administered to cancer patients, generally using one of the following dosage regimens for intravenous and intracavitary administration: (1) 400 mg/kg per day for 5 consecutive days per month or (2) 2 g/kg once a month. For subcutaneous use, the IVIG preparation may be administered in the dose of 4 mg/kg per day for 5 consecutive days per month or 20 mg/kg once a month. However, these suggested regimens may be varied according to the patient's age and physical condition, and the severity of disease. The exact protocol will be determined by the treating physician, taking into consideration various factors and circumstances of each patient. Following administration of the gamma globulin preparation, the patient's progress will be monitored according to standard medical procedure. Additionally, the patient will be examined for tumor metastasis or regression.

### Example 7

### Effect of Intravenous Administration of Gamma Globulin to a to Representative Melanoma Patient

A forty-two year old male underwent surgery in September 1989 for the wide excision of a malignant melanoma lesion (depth 1.3 mm) on his left thigh. In May 1991, he underwent a hyperthermic perfusion of the leg with cisplatinum because of a local recurrence of the melanoma in a left femoral lymph node. At that time there was no evidence of metastatic disease in the patient. In February 1993, computerized tomography (CT) scans of the chest and abdomen revealed mass lesions in the spleen (one lesion), the liver (five lesions, the largest being 3×3 cm) and the lungs (four lesions, the largest being 1.5×1.5 cm). Despite the lesions, the patient was asymptomatic. Soon thereafter, a treatment with IVIG was begun. The patient was intravenously administered IVIG (Miles) at a dose of 400 mg/kg/day for 5 consecutive days per month. After five cycles of treatment, all of the spleen and liver metastases disappeared and there was also a slight reduction in the lung lesions. Afterwards, the patient's condition deteriorated with the appearance of new bone and subcutaneous lesions. He continued to receive IVIG with minor reemergence of liver metastases. The patient expired after receiving 12 cycles of IVIG.

As will be appreciated by one of skill in the art, large liver and spleen metastases of melanoma do not regress spontaneously. Furthermore, it is well known that after the detection of such large metastases, the survival time of patients is usually no longer than a few months.

### Example 8

### Effect of Gamma Globulin on T-Cell Lymphoma Cells In Vitro

Materials and Methods

Cell Proliferation Assays

The rat Nb2-llC T-cell lymphoma cell line was used) Pines et al., “Inhibition of the proliferation of Nb2 cells by femtomolar concentrations of cholera toxin and partial reversal of the effect by 12-0-tetradecanoyl-phorbol-13-acetate,” J. Cell. Biochem., 37, pp. 119-129 (1988)). Cells (1.2×10⁴/well) were incubated with RPMI medium containing 5% fetal bovine serum (FBS) in 96 microtiter plates for 48 h. IVIG (Sclavo S. p. A., Siena, Italy) was added to the cell cultures at concentrations of 50,25,10 and 5 mg/ml for 48 and 120 hours. Cultures containing cells suspended in RPMI medium and 5% FBS served as controls.

During the last 18 hours of incubation, each well was pulsed with 1 μCi [³H]-thymidine. The cells were harvested and the [³H]-thymidine uptake was determined in a LKB liquid scintillation counter (LKB, Piscataway, N.J., USA).

Cell Cycle Analysis

Flow cytometric analysis of the cell cycle of Nb2-llC cells was carried out by propidium iodide staining. A sub-G1 peak by FACS analysis represents the apoptotic cells and the percentage of these among the whole cel population can be analyzed. Cells, at a concentration of 1.2×10⁵ were cultured in RPMI medium and IVIG at a concentration of 50, 25, 10 and 5 mg/ml and incubated for 48 h at 37° C. Cells cultured in RPMI and 5% FBS served as control. At the end of the incubation period, the cells were washed three times with PBS and resuspended in staining buffer containing 0. bovine serum albumin (BSA), 50 g/ml propidium iodide, 0.1% Triton X-100 and 1 mg/ml RNAase (boiled for 10 min). Samples were examined after 30 min of staining on a FACScan flow cytometer (Becton Dickinson & Co., Mountain View, Calif., USA).

Detection of Apoptosis by Acridine Orange Staining

Acridine orange staining was performed according to Hare and Bahler, “Analysis of plasmodium falciparum growth in culture using acridine orange and flow cytometry,” J. Histochem. Cytochem., 34, pp. 215-220 (1986). Cytospin preparations were made directly on slides from Nb2-llC cells treated with 25 mg IVIG. Slides were fixed with 100% ethanol for 10 minutes. Acridine orange at 1. was dissolved in citrate/EDTA buffer (0.13M Na₂HPO4, 0.35M citric acid and 1 M Na2EDTA, pH 6.5) and applied on the slides for 30 minutes. Cells were examined and counted under a fluorescence microscope (Olympus BH-2).

Results

Proliferation of Nb2-llC cells was inhibited in a dose-dependent manner following incubation with various IVIG concentrations. The cell proliferation inhibition was a result of apoptosis rather than a cell cycle arrest, because cell cycle analysis revealed significant levels of apoptotic cells in IVIG treated cultures. The percentage of apoptosis was directly related to the IVIG concentration (i.e., the highest degree of apoptosis was observed at a concentration of 50 mg/ml). These results were confirmed by acridine orange staining, where apoptotic bodies were seen in the IVIG treated culture. In the untreated culture, only well-preserved nuclei were seen.

It will therefore be appreciated that IVIG, at concentrations similar to those found in the blood of patients treated with IVIG, is capable of inducing apoptosis in a T-cell lymphoma cell line.

### Example 9

### Effect of IVIG on the Proliferation, Invasion and Metastatic Capacity of Murine Colon Carcinoma Using In Vitro and In Vivo Model Systems

Material and Methods

Mice

Inbred BALB/c female mice aged 10-12 weeks were purchased from the Animal Breeding Facility at the Sackler Faculty of Medicine at Tel Aviv University. All procedures were reviewed and approved by the Sheba Animal Research Committee.

Cell Line

The CT26 mouse colon carcinoma cell line was obtained from the American Type Culture Collection (ATCC, Manassas, Va.) and maintained in RPMI (Biological Industries, Beit Haemek, Israel) supplemented with 10% bovine serum/HEPES/penicillin/streptomycin /nonessential amino acids sodium pyruvate at 37° C. in an atmosphere of 5% CO₂ and 95% humidity.

IVIG Preparation

IVIG preparation used in the study was kindely provided by Omrix Biopharmaceuticals Inc., Nes-Ziona, Israel.

Tumor Cell Proliferation Assay

The effect of IVIG on cell proliferation was evaluated using an in vitro XTT Cell Proliferation assay Kit (Biological Industries, Beit Haemek, Israel). This assay is based on the cleavage of XTT by metabolic active cells, resulting in an orange formazan dye. The amount of orange formazan dye produced is quantitated using a spectrophotometric plate reader to measure the absorbance at 450 nm. The assay was carried out essentially as described by manufacturer. Briefly, CT26 cells were plated at a low density (5×10³ cells/well) in a 96-well plate with or without different concentration of IVIG. Plates were incubated at 37° C. and 5% CO₂. After 24- to 72 hours 50 μl of the XTT reaction solution was added to each well and after incubation for additional two hours, the absorbance of the samples was measured, and the percentage of inhibition of CT26 cell proliferation was calculated.

Tumor Cell Invasion Using Matrigel Assay

The invasive ability of carcinoma cells was evaluated using Matrigel-coated transwell chamber. CT26 cells (2.5×10⁴) in 750 μl of RPMI 1640 medium containing 7% FCS were placed in the upper wells. After incubation for 48- to 72 hours, cells in the upper wells were removed by wiping with a cotton swab. Number of the cells on the lower surface of filters and the cells that passed through the matrigel-coated membranes to the lower chamber was determined using XTT Reagent kit. The amount of cells that invaded the matrigel, as a percent of total seeded cells was calculated.

Development of In Vivo Lung Metastases

The question of whether or not lung metastasis can be prevented in vivo by injection of IVIG was examined as follows. Eight week-old BALB/c mice, 16-24/group, were injected with 2×10⁴ CT26 cells i.v. On days 0 and 14 after cell inoculation, mice were intravenously injected with either maltose or different concentrations of IVIg. 30 days following the initial tumor challenge, mice were sacrificed and their lungs were harvested en bloc and dissected away from the heart and thymus. The lungs were immediately weighed and/or examined for the presence of metastatic foci. For identification of experimental pulmonary metastases, the trachea of the dead animal was dissected free from surrounding structures and transected. Two ml of India ink solution was injected into the lungs via the trachea until each of the lobes was stained deep black. The lungs and trachea were resected en bloc and placed in Fekete's solution for 24 h. This solution permanently bleached white the tumor foci on the lung surface. The staining procedure results in a clear distinction between metastases (white) and normal lung tissue (black). The presence of tumor nodules in the lungs was confirmed by histology.

Statistical Analysis

The statistical significance of differences among the results obtained was analyzed using the two-tailed, unpaired Student t test. A difference was considered statistically significant when the p value was <0.05.

Results

Firstly the impact of IVIG on the proliferation, invasion and metastatic capacity of murine colon carcinoma was evaluated using in vitro model systems. Intra-assay variations were assessed by performing duplicate measurements and at least three independent experiments.

The Effect of IVIG on Cell Proliferation.

To examine the possible action of IVIG on murine colon carcinoma cells proliferation, CT26 cells were treated with 1, 5, 15, or 40 mg/ml of IVIG for 24-, 48-, or 72 hours. A dose- and time-response curve was obtained by plotting both the IVIG dilution rate and the treated time versus absorbance at 450 nm (FIG. 1). No significant inhibition of proliferation was observed, except for the highest IVIG concentration used (significance at p=0.05), when cells were treated with IVIG for less than 48 hours. In addition, no effect on proliferation was noted when cells were treated with lower concentrations of IVIG (1- and 5 mg/ml) for all indicated time points. On the other hand, the treatment of cells for 48- and 72 hours with IVIG at concentrations of 15 and 40 mg/ml, changed the rate of proliferation and reduced the cell number significantly. As shown, 40 mg/ml of IVIG for 24-, 48- and 72 hours gave rise to 32-, 51- and 57% of inhibition, respectively. Even with 15 mg/ml of IVIG statistically significant inhibition of proliferation by 39.2% and by 45% was observed when cells were incubated for 48- and 72 h, respectively.

The Effect of IVIG on the Invasive Capabilities of Cells

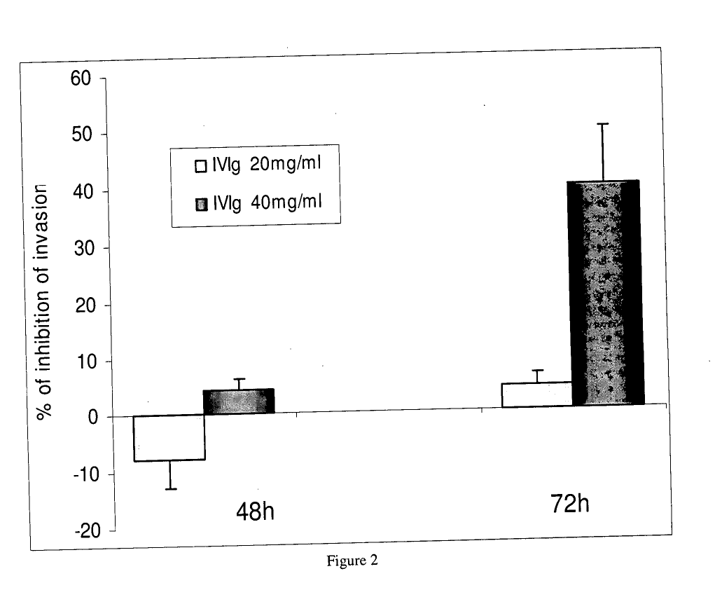

The possibility that IVIG inhibits the invasiveness of CT26 colon carcinoma cells was evaluated in an in vitro experimental metastasis assay using matrigel-coated transwell chambers. Cells were incubated in the presence or absence of IVIG for 48- and 72 hours and bulk that passed through the matrigel-coated membranes into the lower chamber was determined using XTT Reagent kit. The amount of invading cells, as a percent of total seeded cells was calculated. As shown in FIG. 2 the invasive capacity of CT26 cells was decreased in the presence of IVIG. Incubation of tumor cells with 40 mg/ml (30 mg/well) of IVIG for 72 hours resulted in 39.7% inhibition of cell migration across a layer of extracellular matrix. In contrast, after 48 hours IVIG at a concentration of 20 mg/ml (15 mg/well), or at the two tested concentrations did not exhibit a significant effect. Surprisingly a lower concentration of IgG showed improved proliferation inhibition.

The Effect of IVIG on the In Vivo Metastatic Potential of CT26 Cells

To evaluate the anti-metastatic effect of IVIG in vivo, groups of BALB/c mice were inoculated with CT26 tumor cells in 100 μl of PBS, by tail vein injection. The effect of different IVIG doses on the formation of pulmonary metastases was determined four weeks after tumor cell injection.

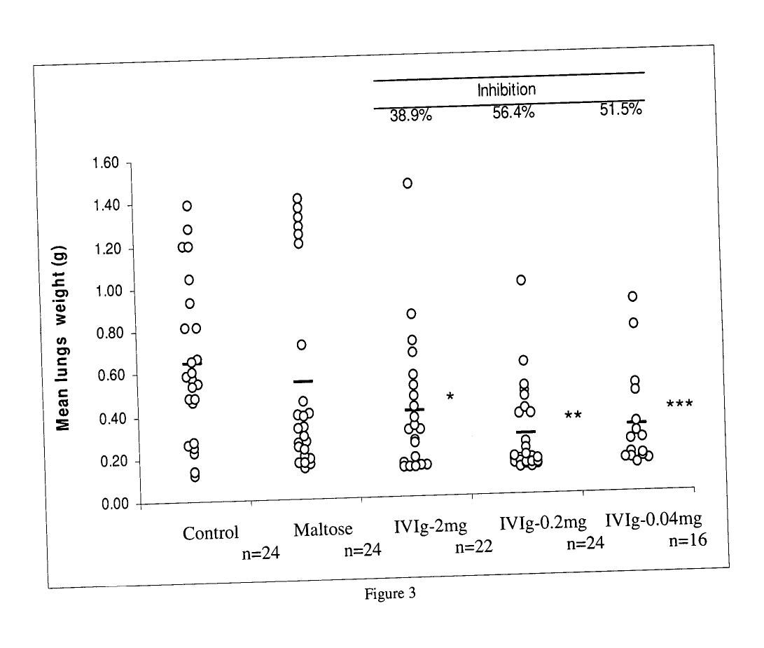

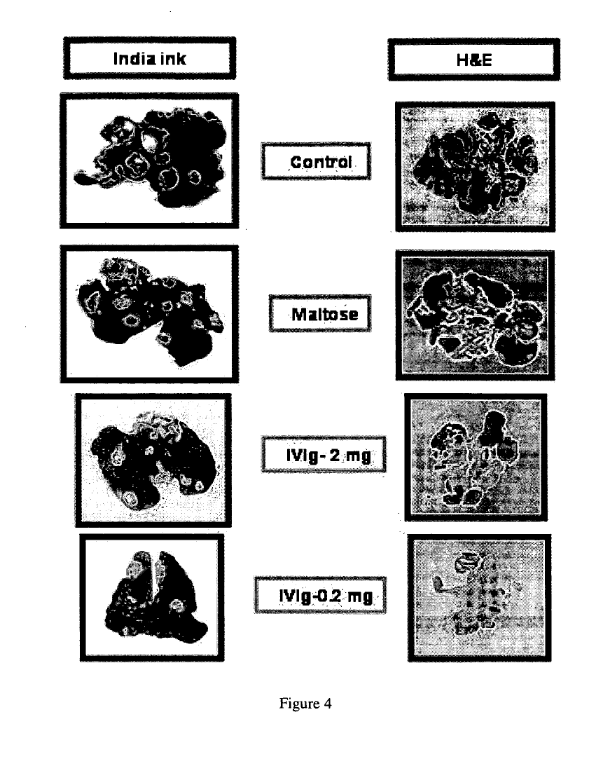

As shown in FIGS. 3 and 4 there was the difference in the number and size of metastatic foci (FIG. 4) in the lungs of the mice treated with IVIG versus untreated mice. This is reflected in the weight difference of lungs among five groups of mice which received different therapies (FIG. 3). Mice exposed to IVIG had a significantly (from p<0.05 to p<0.001) lower mean lung weight than untreated ones (reduction in lung weights by 38.9%, 56.4%, and 51.5% in mice treated with IVIG 2.0, 0.2 and 0.04 mg/mouse, respectively). In addition, no significant reduction in lung weight was observed in animals inoculated with maltose. These results suggest that systemic treatment with IVIG inhibited lung metastatic potential of CT26 cells.

While we have described above specific examples of this invention, it will be apparent to those skilled in the field of cancer therapy that our basic methods may be altered according to need. Therefore, it will be appreciated that the scope of our invention is to be defined by the claims appended hereto rather than by the specific embodiments presented hereinbefore by way of example.

What is claimed is:

1. A method for inhibiting colon cancer, or its metastases in a subject comprising the step of administering to the subject a preparation of IVIG or fragments thereof, thereby preventing tumor cell proliferation, invasiveness or metastasis.

2. The method of claim 1, whereby the preparation of IVIG, or fragments thereof is administered intravasclularly, intracavitarily, intratumorally, intrathecally or subcutaneously.

3. The method of claim 2, whereby the preparation of IVIG is administered at a dosage of between about 0.05 g/kg/month to about 2 g/kg/month.

4. The method of claim 1, whereby the colon carcinoma has metastazied to lungs, liver, brain, bone or a combination thereof.

5. The method of claim 2, whereby the subject is administered at least one other treatment modality, prior to, during or after the administration of the IVIG preparation.

6. The method according to claim 5, whereby the other treatment modality is chemotherapy, immunotherapy, radiation therapy, surgery or a combination thereof.

7. Use of IVIG or fragments thereof in a composition for inhibiting colon cancer, or its metastases in a subject.

8. The use of claim 7 whereby the IVIG fragment is F(ab′)₂, Fc or a combination thereof.
